(19) United States
(12) Patent Application Publication
(10) Pub. No.: US 2010/0178259 A1
(43) Pub. Date: Jul. 15, 2010

(54) REDUCING FIBROSIS USING MATRIX METALLOPROTEINASE INHIBITORS

(75) Inventors: Gary J. Fisher, Ann Arbor, MI (US); John J. Voorhees, Ann Arbor, MI (US)

Correspondence Address: HARNESS, DICKEY & PIERCE, P.L.C., P.O. BOX 828, BLOOMFIELD HILLS, MI, 48303, US

(73) Assignee: The Regents of The University of Michigan, Ann Arbor, MI (US)

(21) Appl. No.: 12/686,092

(22) Filed: Jan. 12, 2010

Related U.S. Application Data
(60) Provisional application No. 61/143,904, filed on Jan. 12, 2009.

(51) Int. Cl. A61K 31/4045; A61K 31/4462; A61P 17/02; A61P 17/16; A61P 17/00; A61Q 17/04

(52) U.S. Cl. 424/59; 514/419; 514/357

(57) ABSTRACT

Methods and compositions to prevent scarring of the skin include a matrix metalloproteinase inhibitor and a vehicle suitable for topical application. Scarring and damage to the epidermis and extracellular matrix may be reduced and/or prevented. Following skin damage, application of the matrix metalloproteinase inhibitor reduces expansion of the extracellular matrix portion of the skin compared to untreated skin.

### CROSS-REFERENCE TO RELATED APPLICATIONS

This application claims the benefit of U.S. Provisional Application No. 61/143,904, filed on Jan. 12, 2009. The entire disclosure of the above application is incorporated herein by reference.

### INTRODUCTION

The present technology relates to methods and compositions to reduce and/or prevent scarring by topical application of matrix metalloproteinase inhibitors, such as an arylsulfonamido-substituted hydroxamic acid.

When injury, disease, or surgery disrupts the normal architecture of body tissues such as the skin, the body instigates a complex cascade of events collectively known as wound healing. Although the capacity of the outer layer of the skin, the epidermis, for regeneration is phenomenal, wound healing of the deeper skin layer, the dermis, is often accompanied by a fibroproliferative response that leads to the formation of a fibrotic scar. The severity of scarring of an individual in response to injury, disease, or surgery is highly variable and depends on multiple factors, such as wound extent, orientation, and infection.

Wound healing is a complicated reparative process that begins with the recruitment of a variety of specialized cells to the site of the wound, and can involve extracellular matrix and basement membrane deposition, angiogenesis, selective protease activity, and re-epithelialization. An important component of the healing process is the stimulation of fibroblasts to generate extracellular matrix. This extracellular matrix constitutes a major component of the connective tissue which develops to repair the wound area.

The connective tissue that forms during the healing process is often fibrous in nature and commonly forms into a connective tissue scar; a process known as fibrosis. Scars are composed of connective tissue that is predominately a matrix of collagen types 1 and 3 and fibronectin. The scar may include collagen fibers in an abnormal organization or it may include an abnormal accumulation of connective tissue. Many scars include both abnormally organized collagen and excess collagen. Scars may be depressed below the skin surface or elevated above the skin surface.

Scarring may result from sunlight exposure and sunburn, which involves skin damage caused by exposure to the sun's rays, especially ultraviolet rays. There are two main types of ultraviolet light, UVA (wavelengths 320-400 nm) and UVB (wavelengths 290-320 nm), which may cause skin damage. On a dose-to-dose basis, UVB is about 1000 times more harmful than UVA. Chronic exposure of unprotected skin to sunlight can induce premature skin ageing, also known as photoaging, which can include scarring, wrinkling, and abnormal pigmentation of the skin.

With respect to wound healing and scarring, it is often desirable to increase the rate of healing for acute wounds (such as penetrative injuries, abrasion, burns including sunburn, nerve damage, and wounds resulting from elective surgery), chronic wounds (such as diabetic, venous, and decubitus ulceration), or for generally healing compromised individuals (for example, the elderly). However, in some instances, the regulation of scar formation is of primary importance and the rate of wound healing is a secondary consideration. Examples of such situations are scars of the skin where excessive scarring may be detrimental to tissue function, particularly when scar contracture occurs (for instance, skin burns and wounds which impair flexibility of a joint). Reduction and/or prevention of scarring of the skin are also important cosmetic considerations.

Thus, there is a need for compositions and methods that reduce and/or prevent scarring of the skin for functional and/or cosmetic purposes.

### SUMMARY

The present technology includes a topical composition for reducing scarring and methods of using such compositions. The composition includes a matrix metalloproteinase (MMP) inhibitor, such as a member of the hydroxamate family (e.g., (2R)—N′-hydroxy-N-[(2S)-3-(5H-indol-3-yl)-1-(methylamino)-1-oxopropan-2-yl]-2-(2-methylpropyl)butanediamide or a salt thereof) or an arylsulfonamido-substituted hydroxamic acid (e.g., N-hydroxy-2(R)[[4-methoxybenzenesulfonyl]-(3-picolyl)amino]-3-methylbutanamide or a salt thereof), and a vehicle suitable for topical application. Scarring of the skin is reduced or prevented by topically applying the therapeutic composition to damaged skin. Skin damage may be the result of penetrative injury, abrasion, burn, sunburn, or elective surgery, or may be the result of a chronic wound. The composition can be used for reducing or preventing scar formation following sunburn, where for example, such methods comprise applying the composition prior to sunlight exposure or applying the composition to sunburned skin in order to prevent subsequent scarring.

In some embodiments, methods of preventing scarring of skin are provided that include topically applying a composition to skin prior to wounding of the skin, the composition comprising a matrix metalloproteinase inhibitor and a vehicle.

In some embodiments, methods of reducing skin fibrosis due to wounding of the skin are provided, where a composition comprising N-hydroxy-2(R)-[[4-methoxybenzenesulfonyl]-(3-picolyl)amino]-3-methylbutanamide or a salt thereof and a vehicle is topically applied to the skin. The composition is applied prior to an expected wound, concomitant with wounding of the skin, or after wounding of the skin. In some aspects, the composition can be applied before wounding, during wounding, and after wounding of the skin. In other aspects, the composition is applied before wounding and after wounding of the skin. For example, the composition may be used prior, during, and/or following a surgical procedure that involves wounding the skin.

The present methods and compositions present several advantages and benefits by preventing and/or reducing scarring of the skin. For example, there are cosmetic advantages to minimizing the appearance of scarring. There are also functional benefits to reducing the extent of fibrosis to maintain flexibility and performance of the skin. Further treatment of the skin or procedures that require manipulation of the skin benefit from a reduction in scarring thickness.

### DRAWINGS

The present technology will become more fully understood from the detailed description and the accompanying drawings.

FIG. 1 is a photograph of a tissue cross-section of mouse skin;

FIG. 2 is a photograph of a tissue cross-section of mouse skin that was exposed to UV irradiation, showing expansion and thickening of the epidermis and extracellular matrix; and

FIG. 3 is a photograph of a tissue cross-section of mouse skin that exposed to UV irradiation and treated by topical application of MMI270 (i.e., N-hydroxy-2(R)-[[4-methoxybenzenesulfonyl]-(3-picolyl)amino]-3-methylbutanamide), showing less expansion and thickening of the epidermis and extracellular matrix in comparison to FIG. 2.

The figures are for illustrative purposes only of selected embodiments and not all possible implementations, and are not intended to limit the scope of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Detailed Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the apparatus and systems of this technology and, unless explicitly stated otherwise, are not intended to be a representation that given embodiments of this technology have, or have not, been made or tested.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. In addition, disclosure of ranges includes disclosure of all distinct values and further divided ranges within the entire range.

The present technology includes ways to reduce fibrosis and scarring of skin. Aspects include compositions and methods employing at least one matrix metalloproteinase (MMP) inhibitor with a vehicle suitable for topical application. The composition is applied to skin to reduce expansion of the extracellular matrix portion of the skin which can lead to scar formation. In some cases, the composition is applied prior to an anticipated insult which may damage the skin and in other cases the composition is applied after the skin has received some type of insult that can lead to fibrosis. For example, following sunburn, the extracellular matrix portion of untreated skin undergoes expansion and considerable thickening within the skin that can result in scarring and skin damage. Topical treatment of sunburned skin with the present composition that includes an MMP inhibitor reduces expansion of the extracellular matrix portion of sunburned skin and thereby reduces or prevents scar formation.

The present technology takes advantage the surprising and unexpected effect that MMP inhibitors mitigate expansion of the epidermis and extracellular matrix following an insult that normally leads to fibrosis and scarring. MMPs are responsible for breaking down collagen and other components of the extracellular matrix, thus in a straightforward way, the mitigation of fibrosis is counterintuitive. One might predict that inhibition of MMPs would lead to an inability to turnover the epidermis and extracellular matrix thereby resulting in an increase in thickness of these layers, not the reduction observed when MMP inhibitor is applied to the skin. The effect of the present technology is hence opposite of what one might expect.

Scarring can be a gradual process that may progress for days, weeks or months. Scarring begins once an insult to the skin begins to heal, such as closure of a wound. Treatment to prevent scarring can start soon after the insult or wound closure and can continue for days or weeks and may continue for months if necessary. In some cases, treatment may begin prior to an anticipated insult that could lead to scarring of the skin, for example, where an individual is expecting exposure to sunlight that could lead to sunburn. Frequency of treatment may include continuous exposure of the skin to the composition, for example, via a patch or reservoir device applied to the skin, or may include frequent application (e.g., at least once daily) of a topical formulation, including where the composition is applied as needed, or an application frequency directed by a physician. Dosing may include from about 0.01% to about 5% of the MMP inhibitor suspended or solublized in a topical vehicle. Examples of vehicles include various lotions, ointments, or creams.

Scarring of the skin refers to an abnormality in one or more of color, contour (bulging/indentation), rugosity (roughness/smoothness) and texture (softness/hardness), arising during the human skin healing process. Preventing scarring refers to an adjustment to the extent of development of scarring, whereby one or more of the color, contour, rugosity, and texture of the healed skin surface approximates on ordinary visual inspection that of the patient's normal skin. Reducing scarring refers to an adjustment to the extent of development of scarring, whereby one or more of the color, contour, rugosity, and texture of the healed skin surface approaches measurably closer that of the patient's normal skin. Skin includes all surface tissues of the human body and subsurface structure, including mucosal membranes and eye tissue, as well as ordinary skin. A wound is any skin lesion or insult to the skin capable of triggering a healing process which may potentially lead to scarring, and includes wounds created by injury, wounds created by burns and sunburn, wounds created by disease, and wounds created by surgical procedures.

The present compositions and methods reduce and/or prevent scarring of the skin for functional and/or cosmetic purposes and may be used to treat scarring caused by trauma, including cuts and abrasions, surgical procedures, such as incisions and skin grafts, and scarring caused by chemical and thermal burns, including severe sunburn. The present compositions and methods may also be used to prevent and/or treat scarring caused by skin stretching, known as stretch marks, due to growth or pregnancy, for example. The present compositions and methods can reduce or prevent formation of hypertrophic scars and keloid scars. Aspects include applying a composition of the MMP inhibitor in a vehicle before, during, or after wounding of the skin.

Matrix metalloproteinases (MMPs) are a family of approximately twenty-seven Zn²⁺ ion-dependent endopeptidases which are involved in the proteolytic processing of several components of the extracellular matrix, such as collagens, proteoglycans, and fibronectin. MMPs are implicated in several physiological and pathological processes, like skeletal growth, remodeling, cancer, arthritis, and multiple sclerosis. MMPs enzymatically process many of the molecules present in the extracellular matrix, although they display different propensity for various substrates. However, the most common substrate present in the extracellular matrix is collagen, and collagen may be used as a comparison substrate to differentiate among different classes of MMPs.

On this basis, MMPs may be classified into five main groups. The first group includes collagenases (i.e., MMP-1, MMP-8 and MMP-13), which are able to cleave fibrillar collagen, recognizing the substrate using a haemopexin-like domain. The second group includes gelatinases (i.e., MMP-2 and MMP-9), which enzymatically process various substrates of the extracellular matrix (ECM), such as collagen I and collagen IV. Besides the haemopexin-like domain, these MMPs are characterized by the presence of a collagen binding domain (CBD) located in their catalytic domain, formed of three fibronectin II-like repeats. The third group includes stromelysins (i.e., MMP-3, MMP-10 and MMP-11), which are able to hydrolyze collagen IV, but do not cleave fibrillar collagen I. The fourth group includes matrilysins (i.e., MMP-7 and MMP-26), which lack the haemopexin-like domain and are able to process collagen IV but not collagen I. Finally, the fifth group includes membrane-type matrix metalloproteinases (MT-MMPs; i.e., MMP-14, MMP-15, MMP-16, MMP-17 and MMP-24), which contain at the C-terminus an additional domain, represented by an intermembrane region and short cytoplasmic tail. There are also a few MMPs, such as metalloelastase (MMP-12) and epilysin (MMP-28), which do not necessarily fit into any of these five classes.

Information regarding overall MMP structural organization is located in the RCSB Protein Structure Databank. As of December 2007, the RCSB Protein Structure Databank includes about one-hundred-thirty 3-D structures of MMPs, including ligand-free structures and structures complexed with a specific synthetic or natural inhibitor. The database may be accessed on the internet at [www.rcsb.org/pdb/].

MMP structures show a characteristic fold found in zinc-dependent endopeptidases, which includes three α-helices, four parallel β-sheet strands, and one anti-parallel β-sheet strand. Structural requirements important for achieving high binding affinity and selectivity, of substrates and inhibitors, include: an acidic unit anchored through four contact points, bidentate chelation of Zn²⁺, carbonyl groups for hydrogen bonding, more than two extra units for hydrogen bonds, and a hydrophobic moiety.

Matrix metalloproteinase inhibitors include many groups of compounds. Features that are important for a molecule to be an effective inhibitor of the MMP class of enzymes include: (i) a functional group (e.g. carboxylic acid, hydroxamic acid and sulfhydryl etc.) capable of chelating the active site zinc (II) ion (this may be referred to as zinc binding group or ZBG); (ii) at least one functional group providing a hydrogen bond interaction with the enzyme backbone; and (iii) one or more side chains which undergo effective van der Waals interactions with the enzyme subsites. These features may be realized by a variety of different structural classes of MMP inhibitors, which have been identified by a number of methods including structure-based design and combinatorial chemistry.

Classes of MMP inhibitors include the following: succinyl hydroxamates, including succinyl hydroxamates with a P2′ amino acid residue; non-peptidic succinyl hydroxamates; sulfonamide hydroxamates and related structures; non-hydroxamates, including carboxylic acids and N-carboxyalkyl ZBGs (zinc-binding groups), thiol ZBGs, and phosphorus-based ZBGs; miscellaneous natural products, including tetracyclines, catechin derivatives, pycnidone, futoenone derivatives, and groups of pseudopeptides: actinonin, BE-166278, (Bayu) and matlystatin B; and other natural products: nicotianamine, betulinic acid, glycyrrhetinic acid, and rifampicin.

MMPs and MMP inhibitors also include those described by Aureli et al. “Structural Bases for Substrate and Inhibitor Recognition by Matrix Metalloproteinases,” Current Medicinal Chemistry, 2008, 15, 2192-2222; Verna et al. “Matrix metallopoteinases (MMPs): Chemical-biological functions and (Q)SARs,” Bioorganic & Medicinal Chemistry 15 (2007) 2223-2268; Cheng et al. “Role of Sulfonamide Group in Matrix Metalloproteinase Inhibitors,” Current Medicinal Chemistry, 2008, 15, 368-373; and Kontogiorgis et al. “Matrix Metalloproteinase Inhibitors: A Review on Pharmacophore Mapping and (Q)Sars Results,” Current Medicinal Chemistry, 2005, 12, 339-355; which are incorporated herein by reference.

Examples of MMP inhibitors further include: (2R,3R)-3-(cyclopentylmethyl)-N-hydroxy-4-oxo-4-(piperidin-1-yl)-2-[(3,4,4-trimethyl-2,5-dioxoimidazolidin-1-Amethyl]butanamide, known as Cipemastat (Trocade™) and N-[(2R)-2-(Hydroxamidocarbonylmethyl)-4-methylpentanoyl]-L-tryptophan methylamide, known as Ilomastat (Galardin™). Further representative MMP inhibitors include Marimastat (British Biotech), Ro 32-35555 (Roche), CGS27023 (Ciba-Geigy/Novartis), AG3340 (Agouoron), and N-substituted D,L homocysteine hydroxamic acids. For example, succinic acid-based MMP inhibitors include Batimastat, Marimastat, Ro 32-3555, and D-2163/BMS-27591. Sulfonamide/sulphone-based MMP inhibitors include Prinomastat and RS130830. And acyclic a-sulfonamide hydroxamates can inhibit MMP-1.

MMP inhibitors may be derived from natural sources. Examples of MMP inhibitors derived from natural sources include: ellagic acid, apigenin, pomiferin, sappanone, rotenonic acid methyl ether, celastrol, dihydrocelastrol, dihydrogambogic acid, decahydrogambogic acid, 4′-hydroxychalcone, clofocto, atranorin, 11-oxoursolic acid acetate, sericetin, and derivatives thereof.

One particular MMP inhibitor is designated GM6001, which, has the chemical formula (2R)—N′-hydroxy-N-[(2S)-3-(5H-indol-3-yl)-1-(methylamino)-1-oxopropan-2-yl]-2-(2-methylpropyl)butanediamide. GM6001 may be in the form of the free base, a hydrate, or a salt. A representation of the chemical structure of GM6001 is as follows:

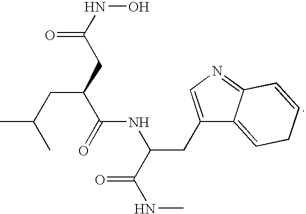

GM6001, also known as Ilomastat and Galardin™, is an MMP inhibitor of the hydroxamate family which binds to the active-site zinc atom present in members of this class of proteinases.

MMP inhibitors also include arylsulfonamido-substituted hydroxamic acids and include the compounds described in U.S. Patent Application Publications 2008/0249032 to Bertini et al. and 2008/0275127 to Breitenstein et al.

One particular arylsulfonamido-substituted hydroxamic acid MMP inhibitor is designated MMI270, which has the chemical formula N-hydroxy-2(R)-[[4-methoxybenzenesulfonyl]-(3-picolyl)amino]-3-methylbutanamide. MMI270 may be in the form of the free base, a hydrate, or a salt, such as a hydrochloride monohydrate salt. Previously, MMI270 was designated CGS27023A and is also known by this name. A representation of the chemical structure of MMI270 is as follows:

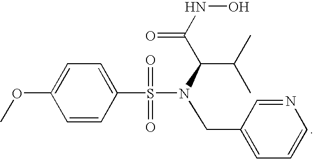

MMI270 is a synthetic hydroxamic acid derivative that can competitively to bind to the Zn²⁺ ion in the active site of a wide range of MMPs, such as MMP-1, MMP-2, MMP-3, MMP-9 and MMP-13, and can inhibit activity of these matrix metalloproteinases at nanomolar concentrations in vitro.

Matrix metalloproteinase inhibitors such as MMI270 have previously been used in cancer therapies, as described by Hidalgo M, Eckhardt S G, “Development of matrix metalloproteinase inhibitors in cancer therapy,” J Natl Cancer Inst. 2001 Feb. 7; 93(3):178-93. In particular, MMI270 is known to possess antitumor activity, as reported by Wood et al., “CGS27023A, a potent and orally active matrix metalloproteinases inhibitor with antitumor activity,” Proc Am Assoc Cancer Res 1998; 39:83. Clinical studies on MMI270 for advanced solid cancer have already been conducted, as reported by Levitt et al., “Phase I and pharmacological study of the oral matrix metalloproteinase inhibitor, MMI270 (CGS27023A), in patients with advanced solid cancer,” Clin Cancer Res 2001; 7:1912-22.

In view of these antiproliferative and antitumor properties, the effect of MMP inhibitors on expansion of the epidermis and extracellular matrix in skin is surprising and unexpected. MMPs are responsible for breaking down collagen and other components of the extracellular matrix and hence the decrease in thickness of the extracellular matrix observed following topical application of an MMP inhibitor such as MMI270 is counterintuitive. One might predict that inhibition of MMPs would lead to an inability to turnover the epidermis and extracellular matrix thereby resulting in an increase in thickness of these layers. Instead, the observed result is quite the opposite of what one might expect, where application of the MMP inhibitor leads to reduced skin thickness and a reduction or prevention of scarring.

The various MMP inhibitors may be used singly or in combination. The MMP inhibitor may be present in the form of a free base, a hydrate, or a salt. Formulations of the MMP inhibitor and vehicle can also include the MMP inhibitor as an amorphous solid, crystalline form, or solublized form. One or more of these forms may be present in one or more phases of the composition, such as where the composition is an emulsion or dispersion.

The following experiments illustrate embodiments of the present technology. Experimental mice (HRS/J hairless mice) are treated with UV irradiation at a dosage that results in sunburn and consequent scarring of the skin. UV exposure includes daily exposure (Mon-Fri), or 3× per week (Mon, Wed, Fri), at 200-400 mJ/square cm, until scarring occurs, typically after 2-3 weeks. The UVB/A2 source is FS tubes filtered with Kodacel to remove wavelengths below 290 nm. Non-irradiated mice serve as non-wounded controls. The MMP inhibitor MMI270 is applied to a group of mice following UV irradiation and another group of mice is left untreated to act as a fibrosis and scarring positive control. Application of MMI270 used a composition of 1% MMI270 in a vehicle of 70% ethanol (95%) and 30% propylene glycol that was applied daily after UV exposure. The effects of no treatment and topical treatment of MMI270 on skin are compared to each other and to non-irradiated skin.

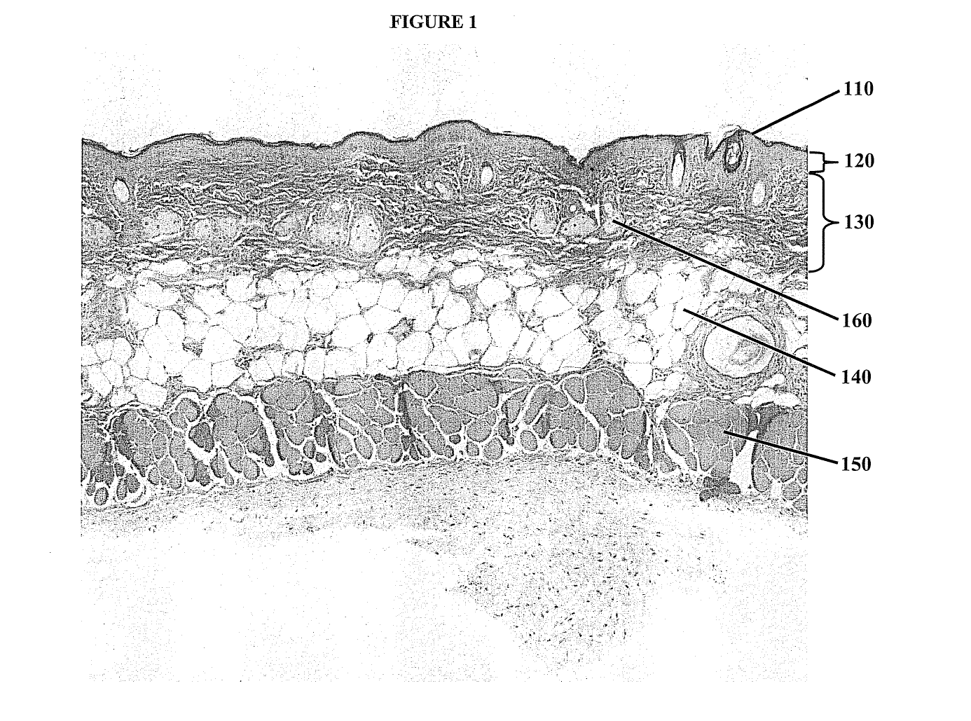

FIG. 1 is a photograph of a tissue cross-section of non-irradiated mouse skin. The outer surface of the skin includes a layer 110 of dead skin cells overtop the epidermis 120. Located beneath the epidermis 120, is a region comprising extracellular matrix 130, which includes collagen and other proteins. A layer of fat cells 140 underlies the extracellular matrix 130 and is followed by muscle 150. Hair follicles 160 can be seen embedded within the extracellular matrix 130 region.

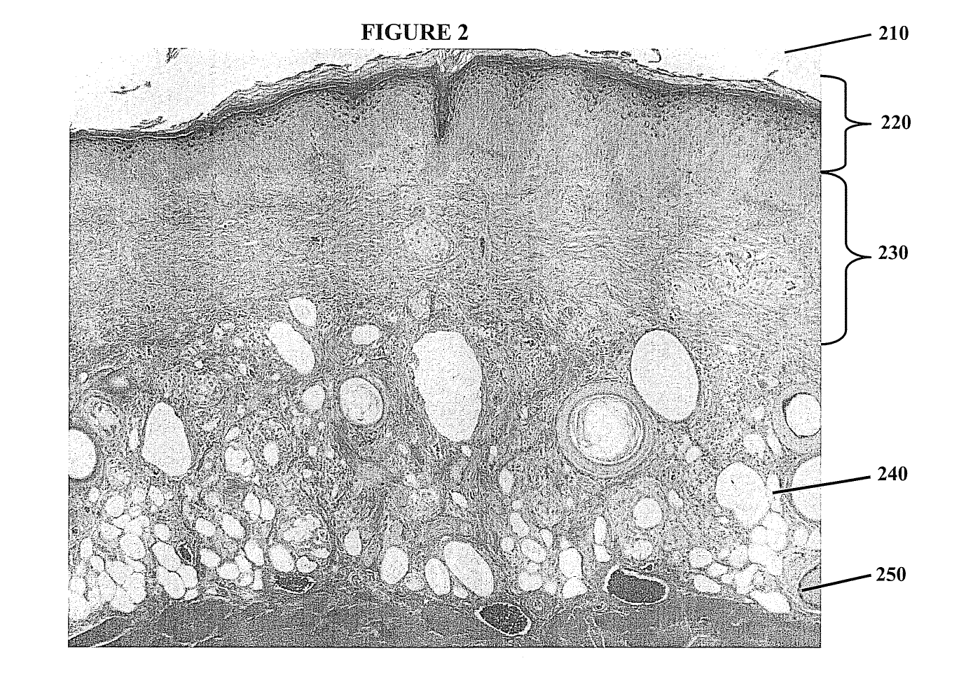

FIG. 2 is a photograph of a tissue cross-section of mouse skin that was exposed to UV irradiation to cause a burn and that was not treated with MMP inhibitor. Morphology of the skin changes in response to the burn wound which can subsequently form into a scar. The outer skin surface and layer 210 of dead skin cells overlies a thickened epidermis 220 followed by a thickened region of extracellular matrix 230. Fat cells 240 and muscle 250 are visible underlying the extracellular matrix 230.

Comparing FIGS. 1 and 2, the epidermis and extracellular matrix show considerable expansion in thickness following UV irradiation and formation of the sunburn wound. This expansion is associated with fibrosis and scarring of the skin.

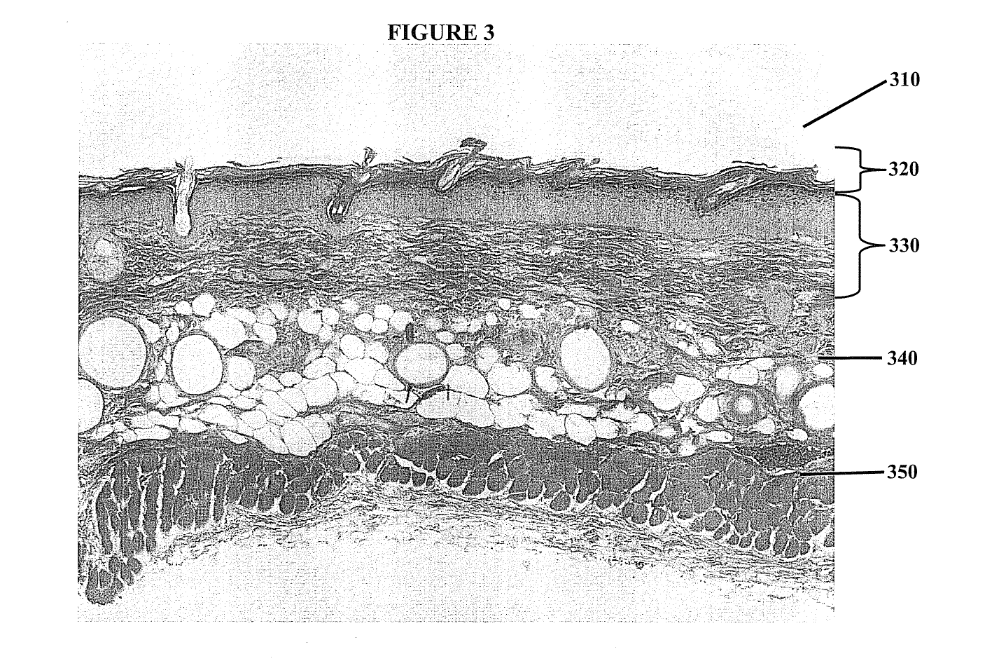

FIG. 3 is a photograph of a cross-section of mouse skin exposed to UV irradiation that was treated by topical application of the MMP inhibitor MMI270 (i.e., N-hydroxy-2(R)-[[4-methoxybenzenesulfonyl]-(3-picolyl)amino]-3-methylbutanamide). An outer dead skin layer 310 covers the epidermis 320 and the region of extracellular matrix 330. Fat cells 340 underlie the extracellular matrix 320, followed by muscle 350.

Comparing FIGS. 2 and 3, the epidermis and extracellular matrix show less expansion in the skin treated with MMP inhibitor. In effect, the MMP inhibitor treated skin in FIG. 3 looks a lot more like the control (non-irradiated) skin shown in FIG. 1. These results demonstrate that topical application of MMP inhibitor reduces the effect of scar formation following wounding of the skin.

The present technology can be employed in several ways. In some embodiments, a method of reducing scarring of the skin comprises topically applying a composition to damaged skin, where the composition includes a matrix metalloproteinase inhibitor and a vehicle. The skin damage may be the result of penetrative injury, abrasion, burn, sunburn, or elective surgery. The topical composition may be applied to the skin damage site daily. The topical composition may also be applied continuously using a patch, bandage, or reservoir device. In some embodiments, a method of reducing scar formation at a skin damage site includes treating the skin damage site with a topical composition until the skin damage is substantially healed, wherein the topical composition comprises a matrix metalloproteinase inhibitor and a vehicle. In some embodiments, a method of decreasing scarring following wound healing comprises administering a topical formulation comprising a matrix metalloproteinase inhibitor and a vehicle to the wound. The topical formulation may be administered to the wound until healing is substantially complete.

The present compositions can employ various vehicles in order to topically deliver the MMP inhibitor. The selected vehicle should be suitable for topical administration to the skin and compatible with the selected MMP inhibitor. Selection and use of suitable vehicles is known in the art and can be adapted as desired.

The vehicle can be a dermatologically/cosmetically acceptable vehicle to act as a diluent, dispersant or carrier for the MMP inhibitor. The vehicle may comprise materials commonly employed in skin care products such as water, liquid or solid emollients, silicone oils, emulsifiers, solvents, humectants, thickeners, powders, propellants and the like. The composition may be in a sprayable liquid form (e.g., a spray that includes the MMP inhibitor in a base, vehicle, or carrier that dries in a cosmetically acceptable way without the greasy appearance that a lotion or ointment would have if applied to the skin). In addition, the compositions contemplated by this invention can include one or more compatible cosmetically acceptable adjuvants commonly used, such as colorants, fragrances, emollients, humectants, and the like, as well as botanicals such as aloe, chamolile, and the like.

The vehicle may be aqueous, anhydrous, or an emulsion, including water-in-oil or oil-in-water emulsions. Water when present will be in amounts which may range from 5 to 99%, from 20 to 70%, and between 40 and 70% by weight. Besides water, relatively volatile solvents may also serve as vehicles. Volatile solvents include monohydric C₁-C₃ alkanols, including ethyl alcohol, methyl alcohol and isopropyl alcohol. The amount of monohydric alkanol may range from 1 to 70%, from 10 to 50%, and between 15 to 40% by weight.

Emollient materials may also serve as cosmetically acceptable vehicles. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes, and polyether siloxane copolymers. The essentially non-volatile polyalkyl siloxanes useful herein include, for example, polydimethyl siloxanes with viscosities of from about 5 to about 25 million centistokes at 25° C. Among the non-volatile emollients useful in the present compositions are the polydimethyl siloxanes having viscosities from about 10 to about 400 centistokes at 25° C.

Among the ester emollients are: (1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate. (2) Ether-esters such as fatty acid esters of ethoxylated fatty alcohols. (3) Polyhydric alcohol esters, including ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. (4) Wax esters such as beeswax, spermaceti, myristyl myristate, stearyl stearate and arachidyl behenate. (5) Sterol esters, of which cholesterol fatty acid esters are examples.

Emollients further include stearyl alcohol, glyceryl monoricinoleate, glyceryl monostearate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, tallow lard, olive oil, palm kernel oil, rapeseed oil, safflower seed oil, evening primrose oil, soybean oil, sunflower seed oil, avocado oil, olive oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, and decyl oleate.

Fatty acids having from 10 to 30 carbon atoms may also be included as a vehicle. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as a vehicle. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5% to 30%, and between 1 and 15% by weight of the composition.

Thickeners may also be utilized as part of the vehicle. Typical thickeners include crosslinked acrylates (e.g. Carbopol 982), hydrophobically-modified acrylates (e.g. Carbopol 1382), taurate polymer, cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums include guar, xanthan, sclerotium, carrageenan, pectin and combinations of these gums. Amounts of the thickener may range from 0.0001% to 5%, usually from 0.001% to 1%, optimally from 0.01% to 0.5% by weight.

Collectively the water, solvents, silicones, esters, fatty acids, humectants and/or thickeners will constitute the cosmetically acceptable carrier in amounts from 1% to 99.9%, preferably from 80% to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. Total concentration of the surfactant will range from 0.1% to 40%, preferably from 1% to 20%, optimally from 1% to 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

The present composition may also include various optional components. These components include additives such as plasticizers, calamine, antioxidants, chelating agents, as well as sunscreens. Other adjunct minor components may also be incorporated into the compositions. These ingredients may include coloring agents, pigments, opacifiers, and perfumes. Amounts of these other adjunct minor components may range anywhere from 0.001% up to 20% by weight of the composition.

Solvents include ethyl alcohol, propanol, butanol, low molecular weight poly(ethylene oxide), glycerin, propylene glycol, 2-butoxyethanol, amyl alcohol, octanol, decanol, acetone, acetic acid, butyl acetate, methylene chloride, isopropanol, acetone, ethylene glycol monoethyl ether, diethylene glycol monobutyl ether, diethylene glycol monoethyl ether, dimethyl sulphoxide, dimethyl formamide, tetrahydrofuran, amyl lactate, benzyl alcohol, 1,2-dichloropropane, 1,4-butanediol, butyl alcohol, thiodyglycol, 1,2-hexanediol, diacetone alcohol, hexylene glycol, betaphenylethyl alcohol, cyclohexanol, furfuryl alcohol, ethyl benzoate, nicotinic acid, picolinic acid, 3-amyloxy-1,2-propanediol, tetrapropyl urea, tetraethyl urea, 1,1-dipropyl-3,3-diethyl urea, cyclohexanone, acetophenone, propylacetate, diethylmalonate, pyridine-2-carbinol and the like;

Powders, such as chalk, talc, fullers earth, kaolin, starch, gums, colloidal silica, sodium polyacrylate, tetra alkyl and/or trialkyl aryl ammonium smectites, chemically modified magnesium aluminum silicate, organically modified montmorillonite clay, aluminum silicate, fumed silica, carboxyvinyl polymer, sodium carboxymethyl cellulose, ethylene glycol monostearate.

The composition may be provided in any suitable physical form for topical application, for example as low to moderate viscosity liquids, lotions, milks, mousses, sprays, gels, foams, aerosols, and creams. These compositions may be produced by procedures well known to the skilled artisan. The cosmetic compositions can be used in various manners as other known compositions in the art including but not limited to various rinse-off and leave-on applications such as shampoos, skin cleansers, skin lotions, conditioners, and mousses.

The composition can be packaged in a suitable container to suit its viscosity and intended use. For example, a lotion or fluid cream can be packaged in a bottle or a roll-ball applicator or a propellant-driven aerosol device or a container fitted with a pump suitable for hand or finger operation. When the composition is a cream, it can simply be stored in a non-deformable bottle or squeeze container, such as a tube or a lidded jar.

The composition may further include a propellant for application to the skin through a spray device, such as an aerosol can. Propellants include trichlorofluoromethane, dichlorodifluoromethane, dichlorotetrafluoroethane, monochlorodifluoromethane, trichlorotrifluoro etane, propane, butane, isobutane, dimethyl ether, carbon dioxide, nitrous oxide.

When used topically, the MMP inhibitor is used preferably at concentrations of between about 0.05% and about 5%, more preferably between 0.1% and 1%.

The embodiments and the examples described herein are exemplary and not intended to be limiting in describing the full scope of apparatus, systems, and methods of the present technology. Equivalent changes, modifications and variations of some embodiments, materials, compositions and methods can be made within the scope of the present technology, with substantially similar results.

What is claimed is:

1. A method of reducing scarring of skin comprising topically applying a composition to wounded skin, the composition comprising a matrix metalloproteinase inhibitor and a vehicle.

2. The method of claim 1, wherein the matrix metalloproteinase inhibitor is (2R)—N′-hydroxy-N-[(2S)-3-(5H-indol-3-yl)-1-(methylamino)-1-oxopropan-2-yl]-2-(2-methylpropyl)butanediamide or a salt thereof.

3. The method of claim 1, wherein the matrix metalloproteinase inhibitor is an arylsulfonamido-substituted hydroxamic acid.

4. The method of claim 3, wherein the arylsulfonamido-substituted hydroxamic acid is N-hydroxy-2(R)-[[4-methoxybenzenesulfonyl]-(3-picolyl)amino]-3-methylbutanamide or a salt thereof.

5. The method of claim 1, wherein the composition includes about 0.01% to about 5% of the matrix metalloproteinase inhibitor.

6. The method of claim 1, wherein the wounded skin is the result of penetrative injury, abrasion, burn, sunburn, or elective surgery.

7. The method of claim 1, wherein the wounded skin is the result of a chronic wound, including diabetic, venous, and decubitus ulceration.

8. The method of claim 1, wherein the composition is applied to the wounded skin daily.

9. The method of claim 1, wherein the composition is applied to the wounded skin via a patch, bandage, or reservoir device.

10. The method of claim 1, wherein the composition is applied to the wound until healing is substantially complete.

11. The method of claim 1, wherein the vehicle comprises a sunscreen.

12. A method of preventing scarring of skin comprising topically applying a composition to skin prior to wounding of the skin, the composition comprising a matrix metalloproteinase inhibitor and a vehicle.

13. The method of claim 12, wherein the matrix metalloproteinase inhibitor is (2R)—N′-hydroxy-N-[(2S)-3-(5H-indol-3-yl)-1-(methylamino)-1-oxopropan-2-yl]-2-(2-methylpropyl)butanediamide or a salt thereof.

14. The method of claim 12, wherein the matrix metalloproteinase inhibitor is an arylsulfonamido-substituted hydroxamic acid.

15. The method of claim 14, wherein the arylsulfonamido-substituted hydroxamic acid is N-hydroxy-2(R)-[[4-methoxybenzenesulfonyl]-(3-picolyl)amino]-3-methylbutanamide or a salt thereof.

16. The method of claim 12, wherein the composition includes about 0.01% to about 5% of the matrix metalloproteinase inhibitor.

17. The method of claim 12, wherein the topical composition is applied to the skin daily.

18. The method of claim 12, wherein the composition is applied to the skin via a patch, bandage, or reservoir device.

19. The method of claim 12, further comprising reapplying the composition to the skin after the skin is wounded.

20. The method of claim 12, wherein the vehicle comprises a sunscreen.

21. A method of reducing skin fibrosis due to wounding of the skin, the method comprising topically applying to the skin a composition comprising N-hydroxy-2(R)-[[4-methoxybenzenesulfonyl]-(3-picolyl)-amino]-3-methylbutanamide or a salt thereof and a vehicle, wherein the composition is applied prior to an expected wound, concomitant with wounding of the skin, or after wounding of the skin.
